(19) United States
(12) Patent Application Publication
(10) Pub. No.: US 2012/0165946 A1
(43) Pub. Date: Jun. 28, 2012

(54) INTRADISCAL MOTION LIMITING MEMBER AND METHOD OF INSTALLATION THEREOF

(76) Inventors: Sean Suh, Plymouth Meeting, PA (US); Mark Welman, Coatesville, PA (US)

(21) Appl. No.: 13/409,964

(22) Filed: Mar. 1, 2012

Related U.S. Application Data
(62) Division of application No. 12/487,142, filed on Jun. 18, 2009.

(51) Int. Cl. A61F 2/44

(52) U.S. Cl. 623/17.16

(57) ABSTRACT

In an exemplary embodiment, the present invention provides a resilient support member capable of being installed inside an intervertebral disc to provide support to an intervertebral disc that has a compromised annulus fibrosis. In one embodiment, the intradiscal member includes an upper support surface, a lower support surface, and an outer wall. In another embodiment, the intradiscal member includes an inner wall defining a volume. In yet another embodiment, the intradiscal member includes a fastening element. The present invention also provides a method for installing the intradiscal member in the nucleus pulposus region of an intervertebral disc.

### CROSS-REFERENCE TO RELATED APPLICATION

The present application is a divisional application which claims priority to U.S. patent application Ser. No. 12/487,142 filed on Jun. 18, 2009, which is incorporated in its entirety herein.

### FIELD OF THE INVENTION

The present invention relates to the surgical treatment of intervertebral discs in the spine that have suffered from tears in the annulus fibrosis, herniation of the nucleus pulposus and/or significant disc height loss.

### BACKGROUND OF THE INVENTION

The main functions of the spine are to allow motion, transmit load and protect the neural elements. The vertebrae of the spine articulate with each other to allow motion in the frontal, sagittal, and transverse planes. The intervertebral disc is a major link between the adjacent vertebrae of the spine.

The intervertebral discs make up about 20-33% of the lumbar spine length and perform the important role of absorbing mechanical loads while allowing for constrained flexibility of the spine. The disc is composed of a soft, central nucleus pulposus surrounded by a tough, woven annulus fibrosis.

The nucleus pulposus is primarily composed of mucoid material containing mainly proteoglycans with a small amount of collagen and can be characterized as a loose hydrogel. The annulus fibrosus consists of fibrocartilaginous tissue and fibrous protein. The collagen fibers are arranged in layers to form concentric rings around the nucleus pulposus. The layers of collagen fibers are arranged in a generally crisscross fashion which allows the annulus fibrosus to withstand torsional and bending loads.

Repeated loading on the intervertebral discs from spinal movement can initiate circumferential tears in the annulus fibrosus of an intervertebral disc, which gradually form radial tears into the nucleus pulposus resulting in the herniation of the nucleus pulposus and/or significant disc height loss. In addition, excessive loading on the intervertebral disc from spinal trauma can also cause tears in the annulus fibrosis resulting in the herniation of the nucleus pulposus and/or significant disc height loss. Herniation of the nucleus pulposus and/or significant disc height loss reduces the disc's ability to resist compressive loads and can also result in excessive motion in the spine such as excessive extension or flexion, resulting in spine segmental instability. The spine is, thus, more vulnerable to trauma and disease such as stenosis of the intervertebral foramen, nerve root compression, and further disc herniation or disc re-herniation.

As such, there exists a need for an intradiscal member capable of being installed inside an intervertebral disc that provides support to the intervertebral disc that has a compromised annulus fibrosis to preclude tearing or, in the case of an existing tear, preclude further tearing of the annulus fibrosis to prevent the herniation or re-herniation of the nucleus pulposus and/or limit further disc height loss.

### SUMMARY OF THE INVENTION

In an exemplary embodiment, the present invention provides a resilient support member capable of being installed inside an intervertebral disc to provide support to an intervertebral disc that has a compromised annulus fibrosis. In one embodiment, the intradiscal member includes an upper support surface, a lower support surface, and an outer wall. In another embodiment, the intradiscal member includes an inner wall defining a volume. In yet another embodiment, the intradiscal member includes a fastening element. The present invention also provides a method for installing the intradiscal member in the nucleus pulposus region of an intervertebral disc.

Further areas of applicability of the present invention will become apparent from the detailed description provided hereinafter. It should be understood that the detailed description and specific examples, while indicating the preferred or exemplary embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of alternate embodiments of an intradiscal member according to the present invention;

FIG. 2 is a perspective view of one embodiment of the intradiscal member of FIG. 1 shown in an installation process;

FIG. 3 is a front perspective view of one embodiment of the intradiscal member of FIG. 1 shown in an installed position;

FIG. 4 is a rear perspective view of one embodiment of the intradiscal member of FIG. 1 shown in an installed position;

FIG. 5 is a front partial cross-sectional view of one embodiment of the intradiscal member of FIG. 1 shown in an installed position where the adjacent vertebral bodies are in an initial position;

FIG. 6 is a front partial cross-sectional view of one embodiment of the intradiscal member of FIG. 1 shown in an installed position where the adjacent vertebral bodies are in a position subject to loading;

FIG. 7 is a front view of an intervertebral disc with a disc bulge; and

FIG. 8 is a partial perspective view of another embodiment of the intradiscal member of FIG. 1 shown in an installed position.

### DETAILED DESCRIPTION OF THE PREFERRED EMBODIMENTS

The following description of the preferred embodiment(s) is merely exemplary in nature and is in no way intended to limit the invention, its application, or uses.

Looking at FIG. 7, an intervertebral disc 1 is shown between adjacent vertebral bodies 2, 3. Typically, when there is movement in the spine, for example, a bending movement such as flexion, extension, or lateral bending, the adjacent vertebral bodies 2, 3 will move with respect to each other. As a result, the intervertebral disc 1 that connects the adjacent vertebral bodies 2, 3 will be subject to loading. As can be seen in FIG. 7, the adjacent vertebral bodies 2,3 have moved at an angle θ′ with respect to each other, compressing a portion of the intervertebral disc 1 located in the direction of the bend from a first dimension to a second, smaller dimension, z. If the intervetebral disc 1 is a normal, healthy disc, there should be no problem with this spinal movement and loading. However, if the intervertebral disc is compromised and, more specifically, the annulus fibrosis is compromised, there is a chance that the disc will bulge or herniate (identified by reference numeral 4) as a result of the movement and loading. If the disc compromise is significant enough, the disc can also lose height as a result of a hernation as the nucleus pulposes is expelled from the disc and/or from water loss. The present invention provides for a member capable of being installed inside an intervertebral disc to provide support to an intervertebral disc that has a compromised annulus fibrosis. In one embodiment, the intradiscal member can provide support to the intervertebral disc to assist in reducing further tearing of the annulus fibrosis thereby decreasing the chance of a disc bulge, herniation or re-herniation of the nucleus pulposus, and/or limit further disc height loss.

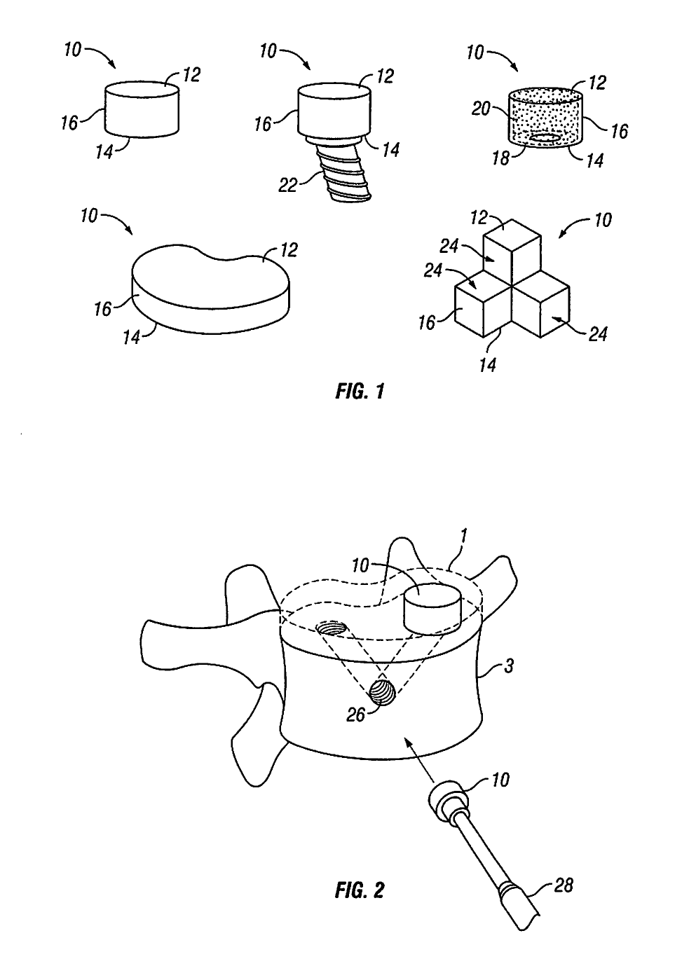

With reference to FIG. 1, several embodiments of an intradiscal member 10 are illustrated. In one embodiment of the intradiscal member 10, the intradiscal member 10 is a resilient, generally solid member comprising at least one biocompatible material such as an elastomeric or polymeric material. However, several polymeric or elastomeric materials may be combined to create the member 10. In one embodiment, the material can be any one of the following: an acrylic based material (having a durometer of about 10 A), a silicon based material (having a durometer of about 25 A to 70 A), or a polyeurathane based material (having a durometer of about 60 A). Preferably, the material used in the intradiscal member 10 will be a material with a resiliency and compressibility that is at least greater than that of the nucleus pulposus. However, other more rigid materials that have little or no resiliency and compressibility may be used, such as titanium or PEEK (polyetheretherketone). In one embodiment, the intradiscal member 10 includes an upper support surface 12, a lower support surface 14, and an outer wall 16. It is contemplated that the intradiscal member 10 can be any number of shapes including spherical, conical, frustoconical, cylindrical, cubic, and polyhedral. It is also contemplated that the intradiscal member can be a more complex or simple shape such as a kidney bean shape.

In another embodiment of the intradiscal member 10, an inner wall 18 can also be included. In this embodiment, the member 10 has an upper support surface 12, a lower support surface 14, an outer wall 16, and an inner wall 18, the inner wall defining an interior 20. In this embodiment, the intradiscal member 10 can be comprised of several materials. For example, outer surfaces as well as the inner wall 18 of the member 10 can be primarily comprised of a polyeurathane jacket while the interior 20 of the member 10 can be filled with silicon. Instead of silicone, the interior 20 of member 10 can be filled with any known biocompatible, resilient and compressible member such as a hydrogel material. Alternatively, the interior 20 can remain empty and serve as an air filled support member.

In another embodiment of the intradiscal member 10, a fastening element 22 is included. In this embodiment, the member 10 has an upper support surface 12, a lower support surface 14, an outer wall 16, and a fastening element 22 extending from the lower support surface 14. The fastening element 22 is configured and dimensioned to serve as an anchor, anchoring the intradiscal member 10 to a predetermined position in the intervertebral disc. Although the fastening element 22 is shown as a shaft having threading extending along at least a portion thereon, any element capable of fastening the member 10 in a predetermined position is contemplated, including a non-threaded interference fit type shaft, a shaft including a plurality of gripping or puncturing elements, or a bonding agent such as glue.

With continued reference to FIG. 1, in yet another embodiment of the intradiscal member 10, the intradiscal member 10 can consist of several discrete components 24. The components 24 at capable of interfacing with each other to form a single member 10. As shown in FIG. 1, the member 10 consists of a plurality of components 24 that are generally cubic in shape and are capable of being arranged in juxtaposition and are capable of being stacked. This variability allows for the member 10 to be created into any desired shape having any desired dimension by using a plurality of identical components 24.

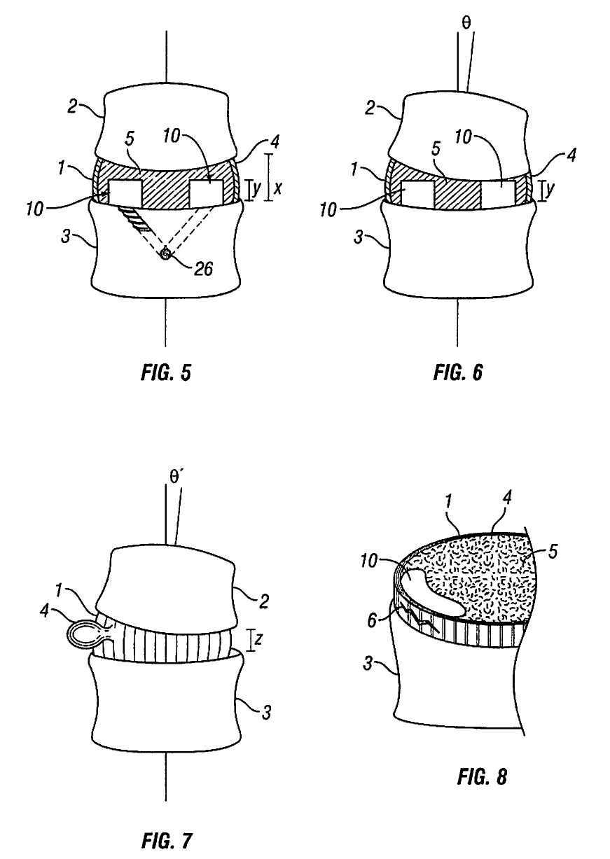

Turning now to FIGS. 2 and 5, the method by which the intradiscal member 10 can be placed in an installed position within the intervertebral disc 1 is shown. It is important to note that the following method is discussed in terms of a single intradiscal member 10, but the following is equally applicable when multiple intradiscal members 10 are used. The surgical approach used to gain access to the intervertebral disc to be treated can be any number of known surgical approaches including an anterior approach, a posterior approach, a translateral approach, or a lateral approach. Once the treatment area is reached, the surgeon can then begin the process of installing the intradiscal member 10.

The intradiscal member 10 can be installed in the intervertebral disc 1 (specifically in the nucleus pulposus 5) via a channel or bore 26 drilled or cut into the vertebral body 3 (or into the vertebral body 2) in the direction of the endplate that is adjacent to the intervertebral disc that is to be treated. The channel 26 preferably extends through the endplate (not shown) of the intervertebral body 3 and into the intervertebral disc 1. The channel 26 is oriented such that the channel 26, when entering the intervertebral disc 1 through the annulus fibrosis 4, will be at or near the predetermined position of installation of the intradiscal member 10 in the nucleus pulposus 5 of the intervertebral disc 1. Alternatively, the channel 26 can be oriented such that the predetermined position of installation is not immediately near the point of entry into the nucleus pulposus 5 but is along the path or trajectory of channel 26 and the predetermined position of installation is further within the nucleus pulposus 5 of the intervertebral disc 1. It is important to note that although the channel 26 is cutting through the annulus fibrosis 4 of the intervertebral disc, it is doing so through the endplate of the vertebral body 3. By cutting through this portion of the annulus fibrosis 4, the intervertebral disc 1 won't be further compromised as the endplate of the vertebral body serves as a natural buttress or barrier. Although the entry or initial opening of the channel 26 can be located anywhere on the intervertebral body 3, in one embodiment, the entry or initial opening of the channel 26 is located centrally on the intervertebral body 3 to prevent compromising the vertebral body 3. Depending on the number of intradiscal members 10 to be implanted and the location of implantation in the nucleus pulposus 5 of the intervertebral disc 1, addition channels can be created, where each channel begins at the same entry initial opening on the vertebral body 3 as the original channel 26 and ends within the nucleus pulposus 5 of the intervertebral body 1 at the desired location for implantation.

Although the channel 26 is shown as being created through the anterior portion of the vertebral body 3 (or vertebral body 2), it is also contemplated that the channel 26 can be created through any portion of the vertebral body 2, 3 including the posterior or lateral portions of the vertebral body 2, 3. For example, it is contemplated that the channel 26 can be created through the pedicle of the vertebral body 2, 3 in the direction of the endplate that is adjacent to the intervertebral disc that is to be treated.

Once the channel 26 is prepared, the intradiscal member 10 can be introduced into the nucleus pulposus 5 of the intervertebral disc 1. The channel 26 is configured and dimensioned so that the intradiscal member 10 can be received through the channel 26 with no change in dimension or a minor change in dimension. In other words, if the channel 26 is configured and dimensions such that the channel 26 is smaller in diameter than the intradiscal member 10, the intradiscal member 10 will compress slightly as it is passed through the channel 26.

The intradiscal member 10 is introduced into the intervertebral disc 1 (specifically into the nucleus pulposus 5 of the disc 1) through the channel 26 with the aid of an installation instrument 28. In one embodiment, the installation instrument 28 will contact and engage the intradiscal member 10 at its distal end. The instrument 28 can then be manipulated to guide the intradiscal member 10 through the channel 26, through the annulus fibrosis 4, into the nucleus pulposus 5 of intervertebral disc 1. Once the intradiscal member 10 is in the desired installation position, the instrument 28 is then disengaged from the intradiscal member 10 and is removed from the intervertebral disc 1 and the channel 26. At this point, if there are additional intradiscal members to be installed, the process is repeated.

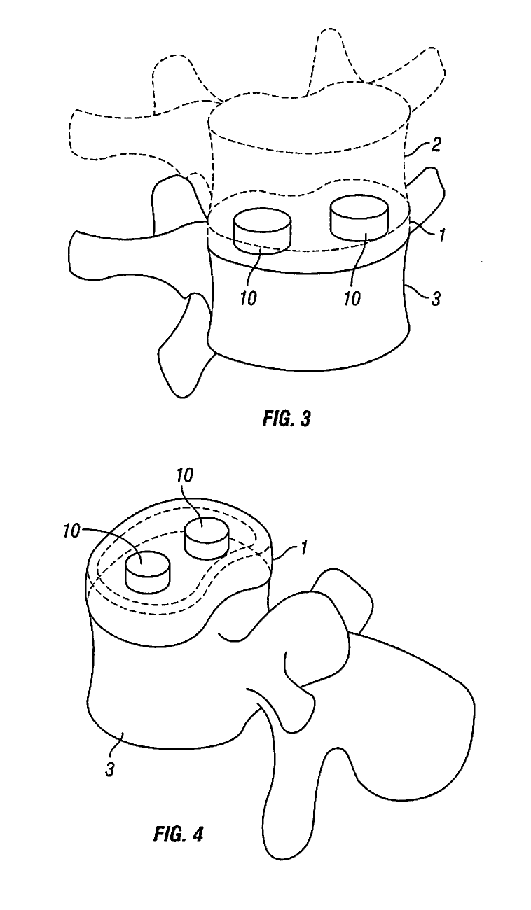

Turning now to FIGS. 3-5, the intradiscal member 10 can be seen in a desired installed position. In one embodiment, the intradiscal members 10 can be anchored in the installed position via the fastening element 22. As discussed above, the fastening element 22 (which can be a threaded shaft, a non-threaded interference fit type shaft, or a shaft including a plurality of gripping or puncturing elements) can anchor the intradiscal members 10 in place by engaging the channel 26 through the annulus fibrosis 4. Alternatively, a bonding agent can be introduced through the channel 26 to anchor the intradiscal member 10 in the installed position. Once the installation and fastening of the intradiscal member 10 is completed, the channel 26 can be filled in with a bone putty or similar void filler.

With reference now to FIGS. 5-7, the intradiscal members 10, when in their installed position, provide support to the intervertebral disc 1 that has a compromised annulus fibrosis 4. In one embodiment, the intradiscal member 10 can provide support to the intervertebral disc 1 to assist in reducing further tearing of the annulus fibrosis 4 thereby decreasing the chance of a disc bulge, herniation or re-herniation of the nucleus pulposus 5, and/or limit further disc height loss. Looking at FIG. 5, the adjacent vertebral bodies 2 and 3 are shown at an initial position where there have been no bending forces introduced. The intervertebral disc 1 is at a first height x and the intradiscal members 10 are at a second smaller height y. In another embodiment, the intradiscal member can be the same height as the intervertebral disc 1, namely a height of x.

Looking next at FIG. 6, bending forces have been introduced moving vertebral body 2 with respect to vertebral body 3 at an angle θ. The portion of the intervertebral disc 1 located toward the direction of the bend compresses while the portion located away from the bend expands. With the intradiscal members 10 implanted, the amount the intervertebral member 1 will compress is limited to about y, the height of the intradiscal member 10. In the embodiment where the intradiscal members 10 are the same height as the intervertebral disc 1, the intervertebral disc 1 may compress slightly or not at all. By limiting the amount of compression of the intervertebral disc 1, the nucleus pulposus 5 will not be pushed against the compromised annulus fibrosis 4 as much (when compared to FIG. 7 where the compressed portion of the intervertebral disc 1 is compressed to a height z which is smaller than either x or y) as a result of the compression of a portion of the intervertebral disc 1 which cause the nucleus pulposus 5 to move towards the expanded portion of the intervertebral disc 1. By limiting the amount of nucleus pulposus 5 being pushed up against a compromised annulus fibrosis 4, the chance of a disc bulge, herniation or re-herniation is reduced.

Turning now to FIG. 8, in another embodiment, the intradiscal member 10 can also function as a barrier to prevent the nucleus pulposus 5 from being expelled through a tear 6 in the annulus fibrosis 4 of the intervertebral disc 1. In one embodiment, the intradiscal member will be configured and dimensioned to be larger than the tear 6 and will be positioned such that the intradiscal member abuts or is in close proximity to the tear 6. By being appropriately sized and situated, the intradiscal member 10 can prevent the nucleus pulposus 5 from being expulsed through the tear 6 in the annulus fibrosis 4 when the intervertebral disc is subject to forces resulting from spinal movement and/or loading.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method of installing a support member within an intervertebral disc comprising:
creating a channel in a vertebral body, the channel extending from an outer surface of the vertebral body through an endplate of the vertebral body;
creating an opening in an annulus fibrosis of the intervertebral disc;
introducing the support member into the channel via an installation instrument; and
placing the support member in a predetermined position in the nucleus pulposus of the intervertebral disc.

2. The method of claim 1, further comprising anchoring the support member in place.

3. The method of claim 1, further comprising filling the channel with a bone void filler.

4. The method of claim 1, further comprising:
creating a second channel in the vertebral body, the second channel extending from the outer surface of the vertebral body through an endplate of the vertebral body, wherein the second channel begins in or near the first channel;
creating a second opening in an annulus fibrosis of the intervertebral disc;
introducing a second support member into the second channel via the installation instrument; and
placing the second support member in a predetermined position in the nucleus pulposus of the intervertebral disc.

5. The method of claim 4. further comprising anchoring the second support member in place.

6. The method of claim 4, further comprising filling the second channel with a bone void filler.

7. The method of claim 1, wherein the support member for supporting an intervertebral disc, the support member comprises an upper support surface; a lower support surface; and an outer wall connecting the upper surface and the lower surface, wherein the support member is installed in the nucleus pulposus of the intervertebral disc, and wherein the resiliency of the support member is less than that of the nucleus pulposus.

8. The method of claim 1, wherein the support member further comprises an inner wall defining an interior volume.

9. The method of claim 1, wherein the support member further comprises a fastening element.

10. The method of claim 9, wherein the fastening element is a shaft having threading extending along at least a portion thereof.

11. The method of claim 9, wherein the fastening element is a bonding agent.

12. The method of claim 9, wherein the fastening element is a shaft having a plurality of protrusions.

13. The method of claim 1, wherein the support member is made of an acrylic based material having a durometer of about 10 A.

14. The method of claim 1, wherein the support member is made from a silicon based material having a durometer of about 25 A to 70 A.

15. The method of claim 1, wherein the support member is made from a polyeurathane based material having a durometer of about 60 A.

16. The method of claim 8, wherein the support member is made from polyeurathane and the interior volume is filled with silicone.

17. The method of claim 1, wherein the support member is any one of the following shapes including generally spherical, generally conical, generally frustoconical, generally cylindrical, generally cubic, and generally polyhedral.

18. The method of claim 1, wherein the support member has a generally kidney bean shape.
